(19) United States
(12) Patent Application Publication
(10) Pub. No.: US 2020/0000361 A1
(43) Pub. Date: Jan. 2, 2020

(54) METHOD AND APPARATUS FOR ACQUIRING MAGNETIC RESONANCE DATA WITH ACCELERATED ACQUISITION OF NAVIGATOR DATA

(71) Applicant: Siemens Healthcare GmbH, Erlangen (DE)

(72) Inventor: Mario Zeller, Erlangen (DE)

(73) Assignee: Siemens Healthcare GmbH, Erlangen (DE)

(21) Appl. No.: 16/456,221

(22) Filed: Jun. 28, 2019

(30) Foreign Application Priority Data
Jun. 28, 2018 (EP) 18180414

(51) Int. Cl. A61B 5/055; G01R 33/483; G01R 33/565

(52) U.S. Cl. CPC A61B 5/055; A61B 2090/374; G01R 33/56554; G01R 33/4835

(57) ABSTRACT

In a method and apparatus for acquiring magnetic resonance data of an acquisition region of a patient, in particular at least a part of the head of the patient, navigator data are acquired for motion correction, between diagnostic data acquisition time windows, in navigator time windows by execution of a fat navigator sequence. The fat navigator sequence has a fat-selective excitation module with at least one radio-frequency pulse and a readout module undersampling in a respective navigator slice. Motion data for the motion correction of the diagnostic data are determined from the navigator data. The navigator data are acquired simultaneously from multiple excited fat navigator slices in the fat navigator sequence using simultaneous multislice imaging, after the excitation module acts on a number of fat navigator slices to be acquired in the readout module.

### BACKGROUND OF THE INVENTION

### Field of the Invention

The present invention concerns a method for acquiring magnetic resonance data from an acquisition region of a patient, in particular at least a part of the head of the patient, by operation of a magnetic resonance device, wherein navigator data are acquired between acquisition time windows for the acquisition of diagnostic magnetic resonance data, in navigator time windows by execution of a fat navigator sequence, having a fat-selective excitation module with at least one radio-frequency pulse and a readout module with undersampling in the acquired navigator slice. Motion data, used for motion correction of the diagnostic magnetic resonance data are determined from the navigator data. The invention further concerns a magnetic resonance apparatus and a non-transitory electronically readable data storage medium that implement such a method.

### Description of the Prior Art

Magnetic resonance imaging represents an image acquisition modality that has become well-established, in particular for medical imaging of patients. In this context it is known that many magnetic resonance sequences, also called acquisition procedures, in general require a relatively long period of time in order to obtain the best possible diagnostic image quality. Movements of the patient cannot be entirely ruled out during this time, such that the anomalies known as motion artifacts constitute an important topic in modern-day magnetic resonance imaging. Motion occurring during the acquisition of magnetic resonance data of an acquisition region of a patient can seriously degrade image quality, and consequently also limit the diagnostic suitability of the data, and even necessitate a new acquisition in extreme cases.

Numerous approaches for avoiding movements and/or for motion correction have been proposed. In addition to sequence-inherent navigators, generally known approaches also include the use of external sensors such as cameras, magnetic field measurements, and so-called “pilot tone” acquisitions. Both prospective and retrospective correction approaches are known in this regard. Furthermore, methods in which magnetic resonance data are triggered by specific respiratory and/or ECG states exist in the prior art. The acquisition times for diagnostic data can be specified by the use of so-called navigators, though it is also conceivable to make use of external sensors. Navigators, in this context, are magnetic resonance acquisitions of comparatively small amount of data, which require only an extremely short amount of time and reveal at least one feature of the patient with sufficient clarity to allow inferences to be made with regard to motion. For example, one-dimensional navigators, in which the position of the diaphragm can be recognized, have been proposed for tracking breathing and/or for respiratory triggering.

Further approaches aimed at reducing motion artifacts in magnetic resonance data include the use of magnetic resonance sequences that are inherently robust against movements, for example radial magnetic resonance sequences and BLADE magnetic resonance sequences, in which the shortest possible measurement time is specified in order to reduce the probability of movements, or which use multiple averaging operations in order to even out motion effects.

Today's simplified user interfaces of magnetic resonance devices in most cases offer the user overall strategies with regard to motion artifacts, for example “high resolution”, “fast” and/or “motion-robust”, where in this instance “motion-robust” strategies often automatically combine the aforesaid approaches for reducing motion artifacts, although there are frequently disadvantages here with regard to the resolution of the resulting magnetic resonance data and/or the speed of the overall acquisition.

Recently, so-called fat navigators have gained in importance as sequence-inherent navigators, i.e. navigators applied between individual diagnostic data acquisition segments (acquisition time windows). This is relevant in particular to the acquisition of magnetic resonance data in the head region of the patient, because there the rare occurrence of fat (spatial “sparsity”) is assured, since fat occurs essentially only in the peripheral regions of the anatomy. Fat navigators known in the prior art employ a fat navigator sequence, which excites spins on a fat-selective basis in a two-dimensional or three-dimensional acquisition region, namely a slice or a thick slab, in an excitation module by one or more radio-frequency (RF) pulses, which means that signals of water-based or water-equivalent spins are suppressed. Binomial pulse sequences, for example, may be used in the excitation module for this purpose.

In an article by Mathias Engstrom et al., “Collapsed fat navigators for brain 3D rigid body motion”, Magnetic Resonance Imaging 33 (2015) 984-991, it is for example proposed to add a fat navigator sequence (“collapsed FatNav”) to a diffusion-weighted 3D multi-slab EPI sequence. Three orthogonal EPI readout modules are used in this case in order to track rigid movements of the head in the image space and perform prospective motion corrections. The actual proposal therein is to use the signal of the leading fat saturation radio-frequency pulse of the diffusion sequence also for the fat navigator, so that only the readout module is required and the overall acquisition time is increased only marginally. However, fat navigators of this type can also be deployed outside of diffusion imaging and, as described by Engstrom et al., use various undersampling (acquired data with sampling that dos not satisfy the Nyquist criterion) schemes, as a result of which ultimately the described “sparsity” of fat in different acquisition regions, in particular in the head region of a patient, is exploited.

With regard to two-dimensional fat navigators, typically a number of slices are acquired within the navigator time window provided for the fat navigator, in order to enable movements to be tracked in all three spatial dimensions. For this reason, only low in-plane resolutions are achievable with two-dimensional fat navigators of the prior art. Three-dimensional fat navigators have the disadvantage that an acquisition is to be performed in three orthogonal directions and it becomes difficult to achieve a sufficiently high spatial resolution, in particular in the k_{z}-direction of k-space, within the available navigator time window. In particular, the so-called “slabs” of the three-dimensional acquisitions are significantly thicker in the z-direction of the scanner coordinate system and often also have a poorer resolution than slices of a slice stack in the case of two-dimensional acquisition variants.

### SUMMARY OF THE INVENTION

An object of the invention is to provide a fat navigator technique for the acquisition of magnetic resonance data that has a sufficiently fine slice resolution, but can nonetheless acquire the navigator data in as short a time as possible.

In a method of the general type described above, according to the invention the navigator data are acquired simultaneously from the fat navigator slices that are excited by the fat navigator sequence, using simultaneous multislice imaging after the excitation module of the navigator sequence acts on a number of fat navigator slices that are to be acquired in the readout module.

The invention therefore uses the known technique of simultaneous multislice (SMS) imaging for the fat navigator. With this technique, multiple slices are excited by a common excitation module within one repetition, and the magnetic resonance signals from those multiple are detected simultaneously. Navigator data of the individual fat navigator slices are subsequently separated from one another algorithmically, using methods such as GRAPPA, for example. Known SMS approaches include, for example, so-called Hadamard coding, methods using simultaneous echo refocusing, methods using wideband data acquisition, as well as methods that use a parallel imaging technique in the slice direction. The last-cited methods include, for example, the blipped-CAIPI technique, as described by Setsompop et al. in “Blipped-Controlled Aliasing in Parallel Imaging for Simultaneous Multislice Echo Planar Imaging with Reduced g-Factor Penalty”, Magnetic Resonance in Medicine 67, 2012, pages 1210-1224.

In slice multiplexing methods of this type, a pulse known as a multiband radio-frequency pulse is used in the excitation module in order to excite two or more slices simultaneously or to manipulate them is some other way, for example to refocus them or, as in the present case of the fat navigator, to selectively saturate/excite them. Such a multiband radio-frequency pulse can be, for example, a multiplex of individual single-slice radio-frequency pulses, which are used to manipulate the individual slices that are to be manipulated simultaneously. In order to be able to separate the resulting magnetic resonance signals of the various fat navigator slices, a different phase is impressed, for example, onto each of the individual radio-frequency pulses prior to the multiplexing, such as by the addition of a linear phase increase, thereby causing the slices to be shifted relative to one another in the spatial domain. As a result of the multiplexing, a baseband-modulated multiband radio-frequency pulse is obtained through summation of the pulse shapes of the individual single-slice radio-frequency pulses.

The use of simultaneous multislice imaging for fat navigators has numerous advantages. Due to the aforementioned “sparsity” of fat, in particular in the case of acquisitions in the head region of a patient, a high multiband factor is enabled, meaning many fat navigator slices can be acquired concurrently. Preferably at least three, and in particular at least eight, fat navigator slices can be acquired simultaneously. In combination with an in-plane undersampling scheme, as described, this enables extremely fast acquisitions of navigator data to be achieved. In this case the in-plane undersampling factor can amount to more than two, in particular to more than ten. When the navigator data are derived from the acquired magnetic resonance signals, a slice-GRAPPA algorithm is then applied initially in order to separate the k-space data of the respective individual slices, after which an in-plane GRAPPA algorithm can be used in order to eliminate aliasing effects. Alternatively, it is also possible to use an algorithm that performs slice separation and in-plane reconstruction in one step.

Compared to conventional three-dimensional fat navigators, the fat navigator slices in accordance with the present invention are actually excited and read out at the same time, i.e. simultaneously. This permits the motion data to be determined in an extremely precise manner and with high resolution, since the period of time available for movements during the acquisition of the navigator data is kept extremely short. It should be noted that it is also possible according to the invention to determine high-resolution motion data in the slice direction/z-direction as well, even without a complete acquisition of the acquisition region, in particular of the head, since thin fat navigator slices can be excited by the SMS imaging and, unlike in the case of three-dimensional fat navigator acquisitions known from the prior art, no slab excitation is performed, which can lead to blurring effects in the case of slices that are infrequently read out. Thus, if a specific required resolution is presumed, significantly shorter fat navigator time windows can be realized by SMS imaging. For example, the navigator data can be acquired in navigator time windows lasting less than 50 ms.

In summary, the present invention is based on acquiring navigator data simultaneously from multiple fat navigator slices by SMS imaging in order to obtain high-resolution data from the fat navigator, even in the case of a lesser slice thickness, and to avoid blurring as in the case of three-dimensional fat navigators, an extremely large number of simultaneously read-out fat navigator slices being possible. Because the fat navigator slices are read out simultaneously, they are in the same motion state, which leads to particularly high-quality navigator data and consequently motion data.

With regard to the SMS imaging and its practical implementation, the method also described in the cited article by Setsompop et al. is preferably used, which means that the fat navigator slices are excited simultaneously, the blipped CAIPIRINHA method is used during the readout, and the navigator data is determined by applying a slice-GRAPPA algorithm and an in-plane GRAPPA algorithm. It should be noted that within the scope of the present invention the calibration data for these or, where applicable, other algorithms applied in order to separate navigator data of individual fat navigator slices or to compensate for the in-plane undersampling can be obtained in different ways. It is conceivable, for example, to acquire calibration data in the first navigator time window, which then possibly lasts somewhat longer, prior to the first acquisition time window and/or to collect calibration data also over a number of navigator time windows, preferably a number of first navigator time windows. In particular when motion data indicating a stronger movement are present, it is furthermore possible to initiate a new acquisition of calibration data and consequently a recalibration of the kernels of the algorithms, in particular the GRAPPA algorithms. Moreover, it is also conceivable to use calibration data of this type also as reference data, in order to achieve a particularly reliable determination of the motion data, as will be explained in more detail below. The use of calibration data from other sources is also conceivable, but less preferable.

In an embodiment of the invention, a binomial pulse sequence is used for fat-selective excitation in the excitation module. The selective excitation of the fat spins thus can be accomplished by multiband binomial pulses, the pulse phases of which are organized such that at the end of the pulse sequence, i.e. the pulse train, only magnetization of fat-equivalent spins is present in the transverse plane, while the magnetization of water-equivalent spins has been folded back into the longitudinal plane. For example, a monopolar 121 binomial pulse sequence can be used, the middle radio-frequency pulse being applied with inverse phase. The idea here is that the first radio-frequency pulse of the binomial pulse sequence deflects the magnetization of both fat spins and water spins by the same flip angle. Both spin types have the same phase at this point in time. Due to the slightly shifted Larmor frequencies for the spin types, a radio-frequency pulse of the binomial pulse sequence relating to twice the flip angle can be applied with a phase of 180° at a second point in time after a time delay. This causes the magnetization of the water spins to be shifted again closer to the longitudinal magnetization, so that after a further time delay, when the spins are once again in phase, the magnetization of the water spins is once again rotated into the longitudinal direction by a final radio-frequency pulse of the binomial pulse sequence, while the magnetization of the fat spins possesses at least transverse components and consequently can be used for imaging.

When many fat navigator slices are acquired simultaneously, which of course is also preferred according to the invention, this can result in extremely strong radio-frequency fields due to many individual radio-frequency pulses overlaying one another (high multiband factor), which may risk the power capacity of the transmit components of the magnetic resonance devices being exceeded, and/or the SAR limit values for the patient being exceeded. In order to counteract this, it is possible to exploit the fact that magnetic resonance signals of fat-equivalent spins have a high intensity, such that an adequate excitation is still assured at smaller flip angles. The flip angle of the binomial pulse sequence can therefore be reduced. For example, a flip angle of less than 30°, in particular of less than 20°, can be used for the entire binomial pulse sequence. Excessively high power levels for individual radio-frequency pulses are avoided in this way.

Alternatively or in addition to such a reduction of the flip angle, other approaches can be adopted in order to keep the power for individual pulses in the excitation module at a low level. In this context, in a further embodiment of the present invention, pulse sequences for different fat navigator slices are radiated offset such that at least one radio-frequency pulse of a binomial pulse sequence is radiated (emitted) between two radio-frequency pulses of the other binomial pulse sequence. This means that the time between two radio-frequency pulses of one binomial pulse sequence is used in order to output pulses of a second binomial pulse sequence so that different fat navigator slices are excited by means of binomial pulse sequences, as has been proposed for example in the post-published German patent application DE 10 2018 201 810.3. That document describes a method for the simultaneous acquisition of magnetic resonance signals from an examination region having two different tissue types in multiple slices that include following steps.

A first binomial pulse sequence is radiated so as to excite at least one first slice, wherein the flip angles of the radio-frequency pulses in the first binomial pulse sequence are chosen such that after the end of the application of the first binomial pulse sequence one of the two tissue types exhibits substantially no transverse magnetization, while the other of the two tissue types exhibits a transverse magnetization. A radio-frequency pulse of the first binomial pulse sequence is radiated at a point in time at which the two different tissue types have an opposite phase angle, and all the radio-frequency pulses of the first binomial pulse sequence are radiated during a switching of magnetic field gradients that gives each a first polarity,

This is followed by radiating a second binomial pulse sequence in order to excite at least one second slice, wherein the flip angles of the radio-frequency pulses in the second binomial pulse sequence are chosen such that after the end of the application of the second binomial pulse sequence, one tissue type exhibits substantially no transverse magnetization, while the other tissue type exhibits a transverse magnetization, and wherein a radio-frequency pulse of the second binomial pulse sequence is radiated in at a point in time at which the two different tissue types have an opposite phase angle. All the radio-frequency pulses of the second binomial pulse sequence are radiated during a switching that gives all of the magnetic field gradients a second polarity. At least one radio-frequency pulse of the second binomial pulse sequence is radiated during the time interval between two radio-frequency pulses of the first binomial pulse sequence.

This is followed by simultaneous readout of the magnetic resonance signals from the at least one first and the at least one second slice.

An approach of said type also proves beneficial with regard to the fat navigator of the present invention.

It should be noted once again that the fat navigator slices of a navigator time window do not have to cover the acquisition region completely, but are preferably chosen as thin, and consequently “undersample” the acquisition region in the slice direction. Highly accurate information is delivered nonetheless in the slice direction on account of the thin slices, the spatial sparsity of fat being exploited to good effect once again.

In exemplary embodiments of the present invention, the motion data are determined by comparison of the currently acquired navigator data with reference data showing a previous motion state, the current navigator data being registered with respect to the reference data. It is particularly advantageous in this case for the reference data to be navigator data of at least one preceding navigator time window.

Thus, in a first, simple embodiment of the present invention, the same fat navigator slices are measured (scanned) in each navigator time window, and registered to the fat navigator slices recorded in the previous navigator time window. In order to obtain maximally accurate motion data in this process, it is particularly advantageous for a large number of fat navigator slices to be acquired simultaneously, in particular more than eight fat navigator slices, which cover the acquisition region as uniformly as possible.

In a preferred embodiment of the present invention, for at least one navigator time window, in particular at least one first navigator time window in a time sequence, a larger of fat navigator slices are acquired for the purpose of generating the reference data. In a version of this embodiment of the present invention, initially, in a first navigator time window, a reference dataset that has a higher number of fat navigator slices is acquired in several repetitions, for example by simultaneous acquisition of four first fat navigator slices in a first repetition and by acquisition of four second fat navigator slices in a second repetition. Fewer fat navigator slices, for example going forward only four, are then acquired in further navigator time windows and in each case are spatially registered with respect to the reference dataset in order to determine the motion data. A more accurate reference is available in this way. It should be noted that in principle it is also conceivable to determine the reference data in the first navigator time window without SMS imaging, also for use as calibration data, for example for calibrating kernels of applied GRAPPA algorithms.

In another embodiment of the present invention, the reference data obtained respectively from a number of navigator time windows succeeding one another in time, in which different fat navigator slices are acquired simultaneously, are combined. From the summation of multiple slice groups of fat navigator slices acquired in successive navigator time windows, it is thus possible to synthesize a reference dataset with respect to which succeeding navigator data are registered in order to determine the motion data. This advantageously avoids an initial scan of longer duration for acquiring reference data, and relaxation effects are attenuated, in particular when fat navigator slices to be acquired are interleaved in an appropriate manner with the imaging slices of the diagnostic acquisition segments. In such an embodiment, the reference data are preferably constantly updated so that the current deviation always relates to the preceding navigator time window. If the acquisition of reference data in a first navigator time window occurs after an excessively strong movement, described by the motion data, it can be beneficial to trigger a new acquisition of reference data.

Even apart from the cited example of the compilation of reference data over a number of navigator time windows, it can be advantageous within the scope of the present invention if, at least at intervals, a different slice group of fat navigator slices is acquired in different navigator time windows, in particular, two groups in alternation. When two different fat navigator slice groups are acquired alternately, these can be interleaved with one another. Recording other slice groups of fat navigator slices in successive navigator time windows has the advantage that relaxation effects are attenuated because the same fat navigator slices are not constantly being excited.

In another embodiment of the method according to the invention, when a reference dataset has been acquired in a first navigator time window, a different slice group of fat navigator slices is always recorded in alternation in the following navigator time windows, and registered to the reference dataset. The slice groups can have fat navigator slices that are in each case offset with respect to one another, i.e. interleaved with one another.

In another embodiment of the present invention, the generation of the initial reference dataset is dispensed with, since different slice groups of fat navigator slices are read out from the acquisition region in different repetitions, in respective navigator time windows. Here as well, for example, an alternating acquisition of interleaved slice groups is possible. The very latest reference dataset, referenced to the acquisition region, can then be derived from two succeeding navigator time windows, whereupon the following navigator data are registered to the very latest reference volume. In addition to the attenuation of relaxation effects, this avoids an initial acquisition of reference data of relatively long duration.

In another embodiment of the present invention, the slice orientation of the fat navigator slices of at least one of the slice groups is rotated with respect to the slice orientation of the fat navigator slices of at least one other of the slice groups, in particular by 90°. Thus, for example, that a different slice orientation of the fat navigator slices is chosen in each navigator time window, which permits a better spatial registration in the case of a (possibly) smaller number of fat navigator slices read out simultaneously. The basis underlying this embodiment is that the fat navigator slices in many cases do not completely cover the acquisition region, so that difficulties in the registration can arise in the case of movements in the slice direction if, for example, no matching reference data are yet available for the now acquired navigator data. However, movements of this type can be excellently and significantly better captured by slices in other directions, which significantly increases the quality of the motion data and consequently its accuracy. Preferably, the different orientations of succeeding navigator time windows can in this instance be incorporated into the registration in each case.

With regard to the acquired diagnostic magnetic resonance data, the motion data can be used for a retrospective or a prospective motion correction thereof. In a retrospective motion correction, a correction is ultimately carried out only when magnetic resonance image datasets are determined from the acquired raw diagnostic magnetic resonance data, to which a motion state is assigned in each case. For example, partial images can be determined for the different motion states of the acquisition region and then merged taking into account the motion data. In a prospective correction, parameters of the magnetic resonance sequence used for the acquisition of the diagnostic magnetic resonance data can be modified directly in order to compensate for motion effects already in advance of the acquisition of the diagnostic magnetic resonance signals, thereby achieving a less laborious and time-consuming evaluation.

The magnetic resonance data are acquired in acquisition time windows lying between the navigator time windows. In this regard, embodiments are conceivable in which only one application of the magnetic resonance sequence, i.e. one temporal repetition, lies between two navigator time windows. Usually, however, it will be possible for a number of repetitions to lie between two navigator time windows. Thus, at least two applications of a magnetic resonance sequence can always take place in the acquisition time windows, i.e. two or more repetitions are performed. Of course, it is also possible to use different magnetic resonance sequences for the acquisition of the magnetic resonance data, particularly when different types of magnetic resonance image datasets of the acquisition region are to be obtained following reconstruction.

Preferably, the magnetic resonance data can be acquired using a magnetic resonance sequence that suppresses fat signals, in particular an EPI (Echo Planar Imaging) sequence. While the navigator method presented herein can basically be combined with all imaging magnetic resonance sequence types, the use of the SMS fat navigators presented here is particularly useful for magnetic resonance sequences which include a fat saturation module, for example EPI sequences or fat-saturated TSE sequences, because the affected fat spins for the imaging are to be suppressed anyway and so negligible crosstalk or negligible magnetization effects should result. In magnetic resonance sequences for the acquisition of the magnetic resonance data which operate without a suppression of the magnetic resonance signals of fat spins, it is possible, as described above, to reduce the flip angle in the excitation module, in particular the flip angle of the binomial pulse sequence, in order not only to reduce the peak power but also to minimize the effect of the fat navigator on the magnetic resonance sequence.

Preferably, the fat navigator sequence is an EPI sequence without refocusing pulses. This means that an EPI readout train accelerated by an in-plane acceleration technique is used, following the excitation of the fat spins in the excitation module. The application of refocusing pulses with high peak power (due to the many fat navigator slices) is thereby avoided. However, other embodiments are conceivable in which, for example, a refocusing is accomplished by PINS pulses.

The present invention also encompasses a magnetic resonance apparatus having a control computer designed to carry out the method according to the invention. All statements made with regard to the method according to the invention apply analogously to the magnetic resonance apparatus according to the invention, so that the cited advantages can also be obtained with this apparatus.

The control computer, which typically has at least one processor and/or at least one memory, can in this case include, in addition to a sequence controller that controls the emission of sequences and the acquisition of magnetic resonance signals, at least one evaluation processor with which the motion data can be determined. Furthermore, a correction processor can be provided so as to implement a prospective and/or retrospective correction of the magnetic resonance data in terms of movements occurring in the acquisition region, in particular the head of a patient.

The present invention also encompasses a non-transitory, computer-readable data storage medium encoded with programming instructions (program code) that, when the storage medium is loaded into a control computer of a magnetic resonance apparatus, cause the control computer to operate the magnetic resonance apparatus so as to implement any or all embodiments of the method according to the invention, as described above.

### BRIEF DESCRIPTION OF THE DRAWINGS

FIG. 1 is a flowchart for the acquisition of navigator data by execution of a fat navigator sequence.

FIG. 2 shows a usable binomial pulse sequence.

FIG. 3 shows the effects of the binomial pulse sequence of FIG. 2 on magnetizations of water and fat spins.

FIG. 4 shows time-staggered switching of binomial pulse sequences.

FIG. 5 is a schematic illustration of a first exemplary embodiment of the method according to the invention.

FIG. 6 is a schematic illustration of a second exemplary embodiment of the method according to the invention.

FIG. 7 is a schematic illustration of a third exemplary embodiment of the method according to the invention.

FIG. 8 is a schematic illustration of a fourth exemplary embodiment of the method according to the invention.

FIG. 9 is a schematic illustration of a fifth exemplary embodiment of the method according to the invention.

FIG. 10 schematically illustrates a magnetic resonance apparatus according to the invention.

### DESCRIPTION OF THE PREFERRED EMBODIMENTS

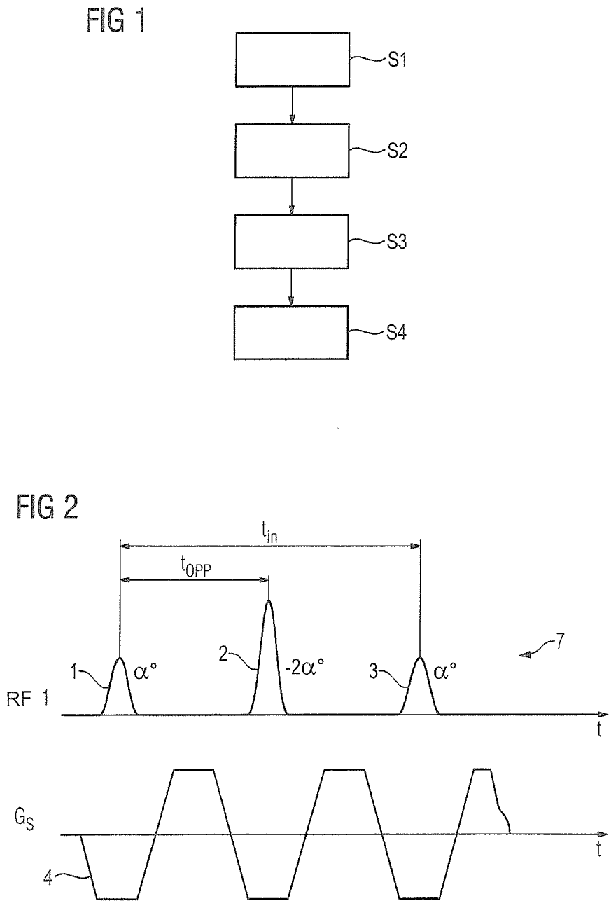

FIG. 1 is a flowchart for acquiring and determining navigator data by execution of a fat navigator sequence in the method according to the invention. In this process, magnetic resonance signals from a number of fat navigator slices are to be acquired simultaneously through the use of SMS imaging in order to determine the navigator data therefrom. For this purpose, in a step S1, a radio-frequency pulse sequence which acts on the number of fat navigator slices and selectively excites fat spins is radiated in an excitation module. For this purpose, in the present example, binomial pulse sequences are used in the excitation module in step S1.

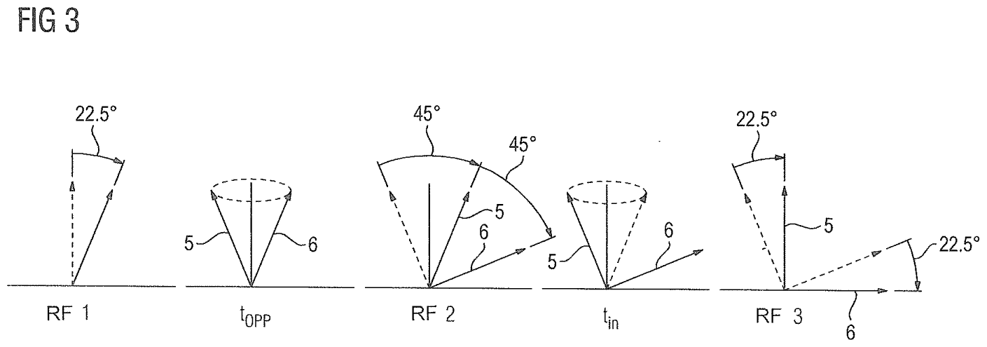

A fat-selective binomial pulse sequence shall be explained in more detail with reference to FIG. 2 and FIG. 3. The binomial pulse sequence 7 shown there is a 1-2-1 binomial pulse sequence, where the specified binomial coefficients relate to the flip angle, α in the figure. Firstly, a radio-frequency (RF) pulse 1 is radiated into the acquisition region, specifically the fat navigator slices, the acquisition region having two different tissue components or spin types, in the present example fat spins and water spins. The first radio-frequency pulse 1 acts both on the fat spins and on the water spins and, as is shown in FIG. 2, deflects the magnetization by the flip angle α, 22.5° in the example, out of the longitudinal direction, the fat spins and the water spins initially still being in phase. After a time delay t_{OPP}, however, the fat and water spins have an opposite phase angle, as is indicated in FIG. 2 by the magnetization 5 and 6, respectively.

If the radio-frequency pulse 2 is now radiated with a doubled flip angle and reversed phase angle, the magnetizations 5 and 6 are tilted further, as shown in FIG. 3. After a further time delay t_{OPP}, i.e. after the time interval tᵢₙ in total, the third radio-frequency pulse 3 is radiated in once again at the flip angle α, such that overall the magnetization vector 5 for water exhibits no transverse magnetization, whereas the magnetization vector 6 for fat lies in the transverse plane.

A binomial pulse sequence 7 of this type, or, as the case may be, the magnetizations 5, 6 resulting after this binomial pulse sequence 7, can then be combined in step S2 with a conventional signal readout, in particular by a readout train of an EPI sequence. Since only the magnetization 6 lies in the transverse plane, the other magnetization 5, in this case the water signal, has no signal component. It should be noted that the waveform 4 of the slice selection gradient G_{S} is also shown in FIG. 3.

For a number of fat navigator slices, in which event, by reason of the spatial sparsity of fat, in particular in acquisition regions relating to the head of the patient, a large number can be chosen, it would in principle be necessary to overlay binomial pulse sequences 7 according to the multiband factor (number of fat navigator slices for a fat navigator sequence), as a result of which excessively high peak power levels can occur, in particular in respect of the central radio-frequency pulse 2. In order to counteract this, two measures can be provided according to the invention. One way is to reduce the flip angle α, so that for example only a smaller total flip angle, for example of 20°, is produced for the magnetization 6 instead of a total flip angle of 90°. Since fat signals have a high intensity, an adequate excitation is also given at smaller flip angles.

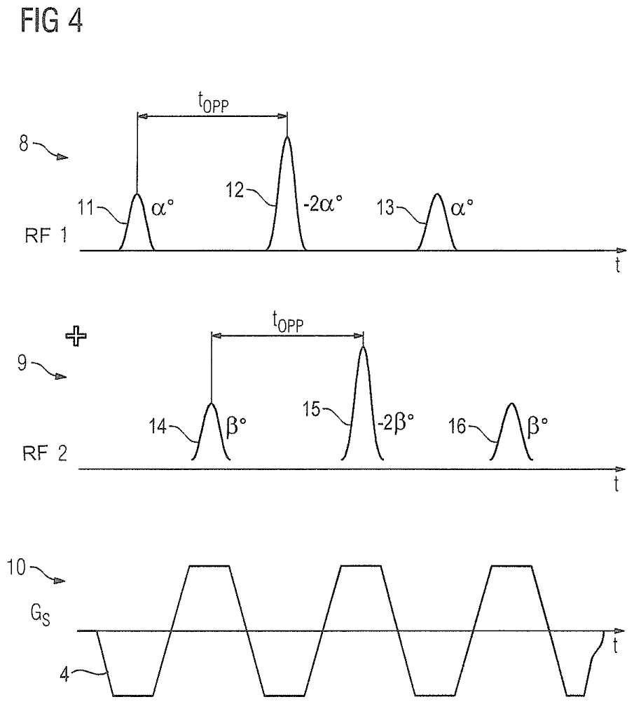

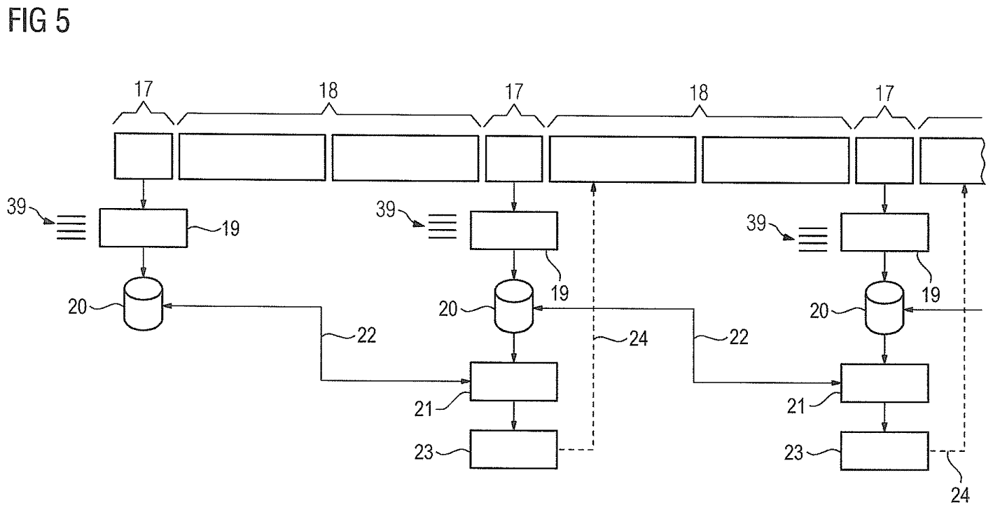

Another way is to use a temporal displacement of radio-frequency pulses which excite different fat navigator slices, as is explained in more detail in relation to FIG. 4.

FIG. 4 shows a first binomial pulse sequence 8 and a second binomial pulse sequence 9, plus, once again, the waveform 10 of the slice selection gradient G_{S}. The first binomial pulse sequence 8 and the second binomial pulse sequence 9 are once again 1-2-1-binomial pulse sequences comprising radio-frequency pulses 11, 12, 13 and 14, 15, 16, respectively, where the first binomial pulse sequence uses a flip angle α, and the second binomial pulse sequence a flip angle β. The time intervals are always chosen such that the magnetization of the water spins after their application in the corresponding fat navigator slices equates to zero. As can be seen, the radio-frequency pulses 11 to 13 and 14 to 16 are switched (activated) offset in time relative to one another, such that with respect to time the radio-frequency pulse 14 comes to lie between the radio-frequency pulses 11 and 12, and the radio-frequency pulse 15 between the radio-frequency pulses 12 and 13. Peak power levels can be further reduced in this way.

In a step S2, cf. once again FIG. 1, a readout module then follows, an EPI readout train accelerated by means of an in-plane acceleration technique being used in the present case in order to avoid refocusing pulses being applied at high peak power levels. In a step S3, the acquired navigator data are then assigned to the various fat navigator slices by application of a slice-GRAPPA algorithm, while in a step S4 an in-plane-GRAPPA algorithm is used with respect to the undersampling in the slice plane. After step S4 navigator data are thus present in the respective fat navigator slices.

FIGS. 5 to 9 show specific embodiments of the method according to the invention during the acquisition of magnetic resonance data by execution of at least one magnetic resonance sequence. The top section in all these figures shows the temporal succession of navigator time windows 17 and acquisition time windows 18. The use of two repetitions in the acquisition time windows is to be understood purely by way of example; it is also possible for more repetitions to take place within the acquisition time windows.

In the first exemplary embodiment shown in FIG. 5, in each navigator time window 17 shown, magnetic resonance signals are acquired simultaneously in the same slice group 39 of fat navigator slices, and the navigator data 20 are reconstructed in respective steps 19, as described in relation to FIG. 1. In this case the navigator data 20 acquired in the respective preceding navigator time window 17 also serves in the present example as reference data, since a registration with the previously acquired navigator data 20 is carried out in each case in step 21 in order to determine the motion data, cf. arrow 22. According to step 23, the thus determined motion data is used for correction purposes, and moreover either for retrospective correction or, preferably, as indicated by the dashed arrow 24, for prospective motion correction, which has immediate repercussions on the imaging in the next acquisition time window 18.

The simple first exemplary embodiment shown here is exceptionally suitable for use when a great number of fat navigator slices is contained in the slice group 39.

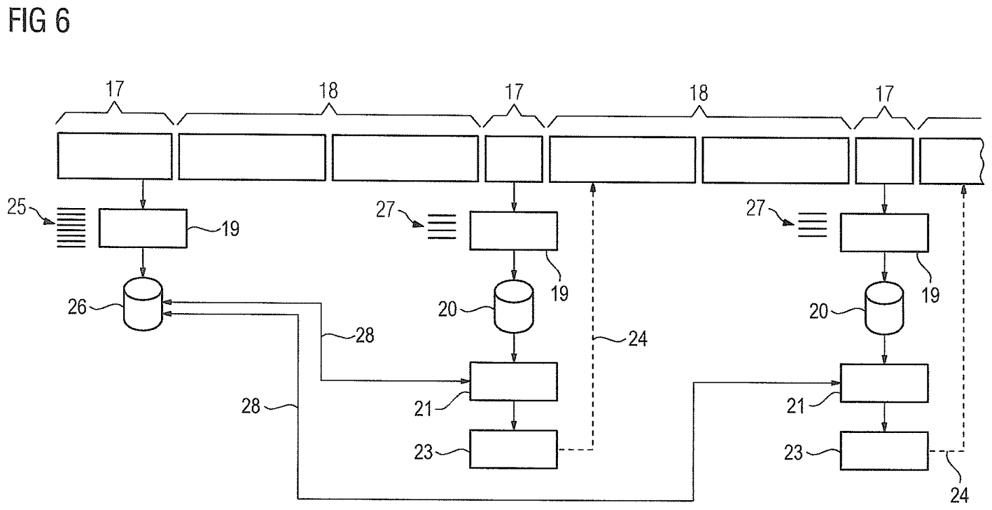

FIG. 6 illustrates a second exemplary embodiment of the method according to the invention, wherein, in contrast to the first exemplary embodiment, in the first navigator time window 17, indicated with extended temporal duration on the extreme left, magnetic resonance signals are acquired in multiple repetitions from a larger number of fat navigator slices of a first slice group 25, from which reference data 26 is determined in step 19. For instance, it is conceivable, in the example of eight navigator slices of the first slice group 25 illustrated by way of example, to perform two SMS imaging repetitions for two slice groups of fat navigator slices that are offset with respect to one another, which together result in the first slice group 25. It is, however, also possible to acquire these without SMS imaging if the reference data 26 is also intended to serve as calibration data, in particular for kernels of the GRAPPA algorithms used.

The reference data 26, which completely cover the acquisition region, therefore also contain information concerning fat components lying between fat navigator slices of the subsequently to be acquired second slice groups 27. This increases the reliability of the registration, performed once again in a step 21, of the navigator data 20 obtained in subsequent navigation time windows 17 with the reference dataset 26 in each case, cf. arrows 28. The additional information in the reference data 26 is moreover useful in particular in the case of movements in the slice direction.

Should excessively strong movements have occurred at a point in time during the overall acquisition time, it is furthermore possible to perform a reacquisition of the reference data 26 in a further extended navigator time window 17.

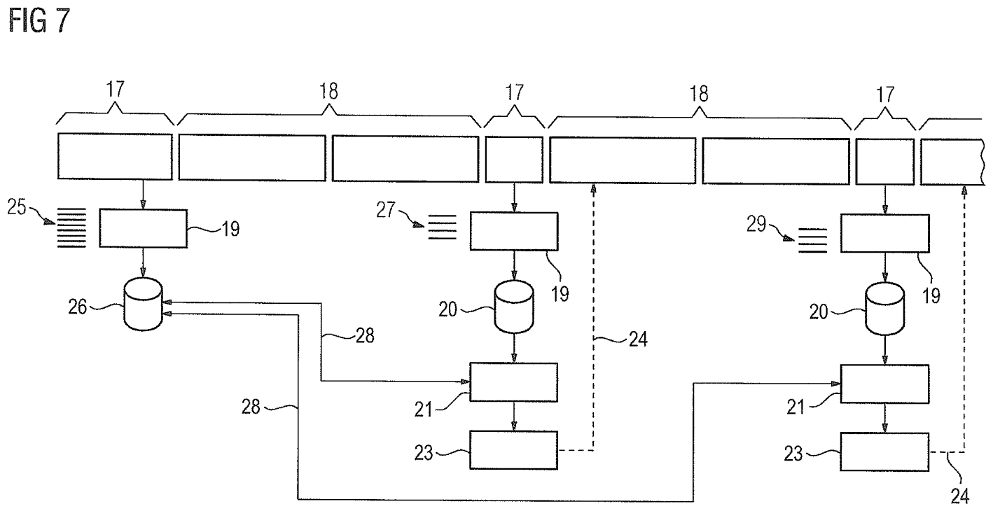

The third exemplary embodiment illustrated by FIG. 7 differs from the second exemplary embodiment in that the same four slices of the slice group 27 are not acquired in each of the further navigator time windows 17, but instead four of the eight slices of the slice group 25 are acquired in alternation in each case, which means that the fat navigator slices of the slice group 27 and the fat navigator slices of the slice group 29 formed from the remaining fat navigator slices of the slice group 25 are acquired alternately. This has the advantage that relaxation effects are attenuated because different fat navigator slices are excited every time.

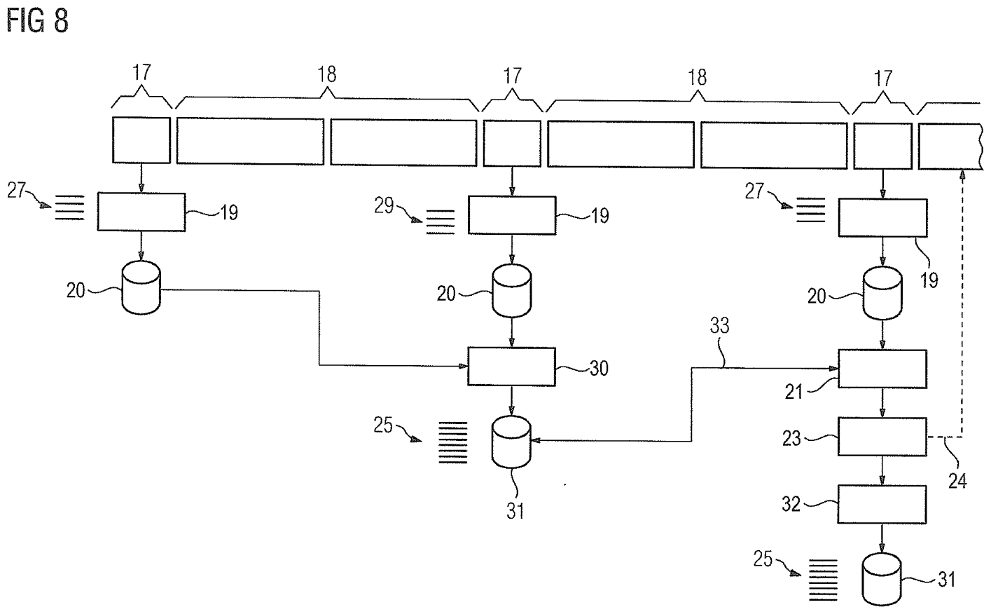

In the fourth exemplary embodiment according to FIG. 8, the slice groups 27 and 29, which can furthermore be understood as interleaved, are likewise measured in alternation, although in that case it is provided to acquire reference data 26, not at the start, but in the second navigation time window 17 when navigator data 20 from both slice groups 27 and 29 is present, to determine from this, in a step 30, reference data for the slice group 25 combining the slice groups 27 and 29. The reference data 31 are updated according to step 32 with each navigation time window 17. Furthermore, the latest reference data 31 at a given time are used for the registration, cf. arrow 33, with the current navigator data 20, in order to enable the motion data to be derived accordingly and a corresponding correction to be performed.

It should be noted that the alternating acquisition of two slice groups 27, 29 is not limiting, so it is also possible to perform passes cyclically through more than the two slice groups 27, 29, for example through three or four different slice groups.

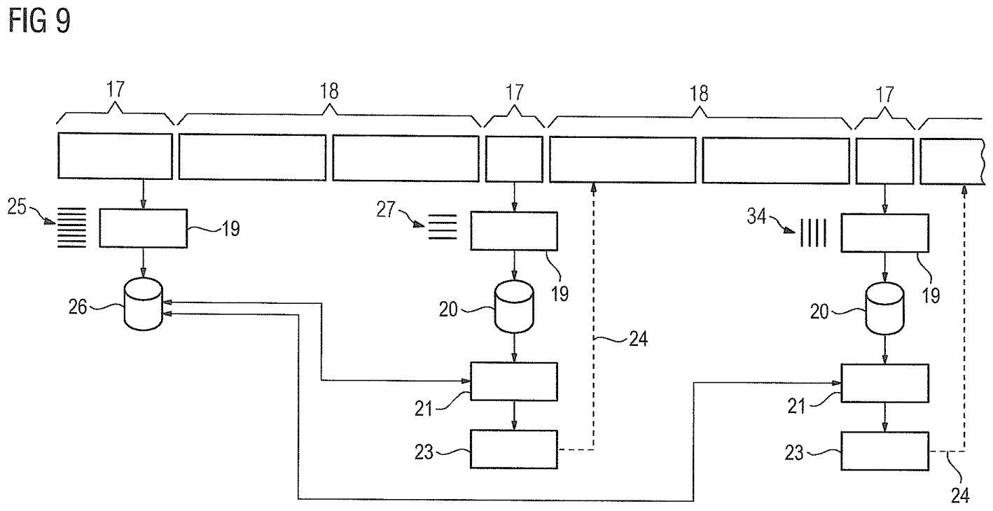

FIG. 9 shows a fifth exemplary embodiment of the method according to the invention, which differs from the third exemplary embodiment in that slice groups 27, 34 having a different orientation of the fat navigator slices are acquired in alternation. This has the advantage of an improved spatial registration, specifically in the case of a smaller number of fat navigator slices that are read out simultaneously.

It should be noted that the embodiment according to FIG. 8, relating to the regular updating of the reference data 31, can, of course, also be combined with the fifth embodiment of FIG. 9.

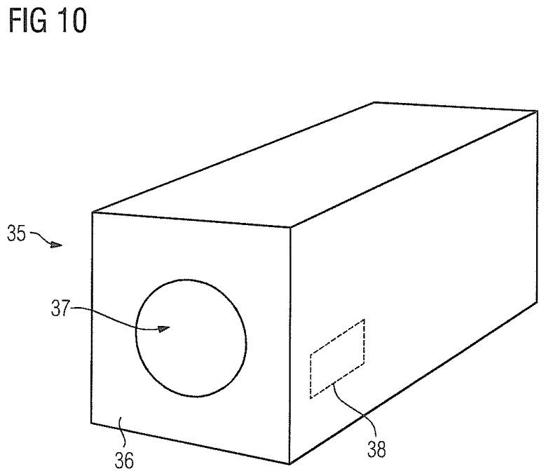

FIG. 10 schematically illustrates a magnetic resonance apparatus 35 according to the invention. This has, as is generally known, an MR data acquisition scanner 36 having a patient receiving zone 37 defined therein, into which a patient can be introduced by a patient table (not shown). A radio-frequency coil array and a gradient coil array are provided in the scanner 36, surrounding the patient receiving zone 37.

The operation of the magnetic resonance apparatus 35 is controlled by a control computer 38, which is also designed to carry out the method according to the invention. For this purpose, the control computer 38 can have an appropriately adapted sequence controller, an appropriately adapted evaluation processor, and a motion correction processor.

Although modifications and changes may be suggested by those skilled in the art, it is the intention of the Applicant to embody within the patent warranted hereon all changes and modifications as reasonably and properly come within the scope of the Applicant's contribution to the art.

1. A method for acquiring magnetic resonance (MR) data from an acquisition region of a patient, said method comprising:
operating an MR data acquisition scanner, while the patient is situated therein, in order to acquire MR diagnostic data from the acquisition region of the patient in respective diagnostic acquisition time windows;
also operating said MR data acquisition scanner in order to acquire navigator data, for motion correction of said MR diagnostic data, from the acquisition region in respective navigator time windows between said diagnostic acquisition time windows, by executing, in each navigator time window, a fat navigator sequence comprising a fat-selective excitation module having at least one radio-frequency (RF) pulse and a readout module in which the navigator data are acquired from respective slices of the acquisition region with undersampling; and
in each navigator time window, acquiring said navigator data simultaneously from a plurality of fat navigator slices that were excited by the fat-selective excitation module, using simultaneous multislice imaging in said readout module, after said fat-selective excitation module has acted on said plurality of fat navigator slices.

2. A method as claimed in claim 1 comprising acquiring said navigator data with said undersampling being defined by an in-plane undersampling factor of more than two.

3. A method as claimed in claim 1 comprising acquiring said navigator data with said undersampling being defined by an in-plane undersampling factor of more than ten.

4. A method as claimed in claim 1 comprising acquiring said navigator data from at least three fat navigator slices simultaneously.

5. A method as claimed in claim 1 comprising acquiring said navigator data from at least eight fat navigator slices simultaneously.

6. A method as claimed in claim 1 comprising acquiring said navigator data from at least eight fat navigator slices simultaneously, with said undersampling being defined by an in-plane undersampling factor of more than ten.

7. A method as claimed in claim 1 comprising operating said MR data acquisition scanner so as to execute a binomial pulse sequence for fat-selective excitation in said fat-selective excitation module.

8. A method as claimed in claim 7 comprising executing said binomial pulse sequence with a flip angle that is less than 30°.

9. A method as claimed in claim 7 comprising executing said binomial pulse sequence with a flip angle that is less than 20°.

10. A method as claimed in claim 7 comprising acquiring respective sets of said navigator data from different simultaneously excited fat navigator slices by emitting said binomial pulse sequence in said MR data acquisition scanner chronologically offset for the respective navigator slices so that one RF pulse of the respective binomial pulse sequence for a respective navigator slice is radiated between respective RF pulses of preceding and following binomial pulse sequences for other navigator slices in the plurality of simultaneously acquired navigator slices.

11. A method as claimed in claim 1 comprising providing the acquired navigator data to a processor and, in said processor, determining motion data from the acquired navigator data with respect to reference data that represents a previous motion state of the acquisition region of the patient, and bringing the acquired navigator data into registration with the reference data.

12. A method as claimed in claim 11 comprising using, as said reference data, navigator data acquired from the acquisition region of the patient in at least one preceding navigator time window with respect to time window for the navigator data from which said motion data are determined.

13. A method as claimed in claim 12 comprising, in said at least one preceding navigator time window, simultaneously acquiring navigator data from a number of fat navigator slices that is larger than a number of fat navigator from which the navigator data are simultaneously acquired in the time window from which said motion data are determined.

14. A method as claimed in claim 12 comprising acquiring said reference data from a plurality of preceding navigator time windows.

15. A method as claimed in claim 1 comprising, in at least some of said navigator time windows, acquiring said navigator data from a different group of navigator slices than in others of said navigator time windows.

16. A method as claimed in claim 15 comprising acquiring said navigator data from two different slice groups that alternate in successive navigator time windows.

17. A method as claimed in claim 15 comprising acquiring the navigator data from two different slice groups that are interleaved with each other.

18. A method as claimed in claim 15 comprising acquiring said navigator data from two different slice groups, with one of said different slice groups having a slice orientation that is rotated with respect to an orientation of another of said slice groups.

19. A method as claimed in claim 1 comprising using said navigator data to produce motion data for motion-correcting said MR diagnostic data by a motion correction technique selected from retrospective motion correction and prospective motion correction.

20. A method as claimed in claim 1 comprising acquiring said MR diagnostic data by executing a magnetic resonance diagnostic data acquisition sequence in which fat signals are corrected.

21. A method as claimed in claim 20 wherein said magnetic resonance diagnostic data acquisition sequence is an EPI sequence.

22. A method as claimed in claim 1 comprising executing, as said fat navigator sequence, an EPI sequence without refocusing pulses.

23. A magnetic resonance apparatus comprising:
an MR data acquisition scanner;
a computer configured to operate said MR data acquisition scanner, while a patient is situated therein, in order to acquire MR diagnostic data from the acquisition region of the patient in respective diagnostic acquisition time windows;
said computer being configured to also operate said MR data acquisition scanner in order to acquire navigator data, for motion correction of said MR diagnostic data, from the acquisition region in respective navigator time windows between said diagnostic acquisition time windows, by executing, in each navigator time window, a fat navigator sequence comprising a fat-selective excitation module having at least one radio-frequency (RF) pulse and a readout module in which the navigator data are acquired from respective slices of the acquisition region with undersampling; and
said computer being configured to operate said MR data acquisition scanner in each navigator time window, so as to acquire said navigator data simultaneously from a plurality of fat navigator slices that were excited by the fat-selective excitation module, using simultaneous multislice imaging in said readout module, after said fat-selective excitation module has acted on said plurality of fat navigator slices.

24. A non-transitory, computer-readable data storage medium encoded with programming instructions, said storage medium being loaded into a control computer of a magnetic resonance (MR) apparatus comprising an MR data acquisition scanner, and said programming instructions causing said control computer to:
operate said MR data acquisition scanner, while a patient is situated therein, in order to acquire MR diagnostic data from an acquisition region of the patient in respective diagnostic acquisition time windows;
also operate said MR data acquisition scanner in order to acquire navigator data, for motion correction of said MR diagnostic data, from the acquisition region in respective navigator time windows between said diagnostic acquisition time windows, by executing, in each navigator time window, a fat navigator sequence comprising a fat-selective excitation module having at least one radio-frequency (RF) pulse and a readout module in which the navigator data are acquired from respective slices of the acquisition region with undersampling; and
in each navigator time window, acquire said navigator data simultaneously from a plurality of fat navigator slices that were excited by the fat-selective excitation module, using simultaneous multislice imaging in said readout module, after said fat-selective excitation module has acted on said plurality of fat navigator slices.
